(19) United States
(12) Patent Application Publication
(10) Pub. No.: US 2022/0370447 A1
(43) Pub. Date: Nov. 24, 2022

(54) METHOD OF TREATING HBV INFECTION USING A CORE PROTEIN ALLOSTERIC MODULATOR

(71) Applicant: Hoffmann-La Roche Inc., Little Falls, NJ (US)

(72) Inventors: Qingyan BO, Shanghai (CN); Valérie COSSON, Richwiller (FR); Sheng FENG, Shanghai (CN); Lu GAO, Shanghai (CN); Yuyan JIN, Shanghai (CN); Annabelle LEMENUEL, Rosenau (FR); Miriam TRIYATNI, Basel (CH); Xue ZHOU, Shanghai (CN); Mingfen ZHU, Shanghai (CN)

(73) Assignee: Hoffmann-La Roche Inc., Little Falls, NJ (US)

(21) Appl. No.: 17/761,521

(22) PCT Filed: Sep. 18, 2020
(86) PCT No.: PCT/EP2020/076065

§ 371 Date: Mar. 17, 2022

(30) Foreign Application Priority Data
Sep. 20, 2019 (CN) PCT/CN2019/107009

(51) Int. Cl. A61K 31/506; A61P 31/20; A61K 31/522; A61K 31/675; A61K 31/7068; A61K 38/21; A61K 31/496

(52) U.S. Cl. CPC A61K 31/506; A61P 31/20; A61K 31/522; A61K 31/675; A61K 31/7068; A61K 38/212; A61K 31/496

(57) ABSTRACT

The present invention relates to method of treating HBV infection in a human patient, wherein the method comprises administration of a therapeutically effective amount of Compound (I), or a pharmaceutically acceptable salt thereof.

The present invention relates to method of treating HBV infection in a human patient, wherein the method comprises administration of a therapeutically effective amount of a core protein allosteric modulator.

### FIELD OF THE INVENTION

Hepatitis B virus (HBV) infection is a major public health problem worldwide, roughly 30% of the world's population show serological evidence of current or past infection. Despite the introduction of a safe and effective prophylactic vaccine against the virus in the early 1980s, it is estimated that there are still more than 257 million chronic HBV carriers worldwide, a high percentage of whom will eventually develop liver cirrhosis or hepatocellular carcinoma (HCC) (WHO Hepatitis B. Fact Sheet N° 204). In the 2010 Global Burden of Disease study (R Lozano, et al. Lancet, 380 (2012), 2095-2128), HBV infection ranked in the top health priorities in the world, and was the tenth leading cause of death (780,000 deaths per year). The sustained clearance of HBsAg has been shown to be protective against disease progression and development of HBV complications including cirrhosis, liver failure, and HCC. Accordingly, HBsAg loss (functional cure) is considered as an optimal endpoint for patients with chronic hepatitis B (CHB). Functional cure is defined as sustained, undetectable HBsAg and HBV DNA in serum, with or without seroconversion to anti-HBs, after completion of a finite course of treatment (Lok AS, Zoulim F, Dusheiko G, et al. Hepatitis B cure: from discovery to regulatory approval. J Hepatol 2017;67:847-861).

Currently, there are two therapeutic classes available for the treatment of CHB: subcutaneously administered Interferon-alfa (IFN-α) preparations (conventional or PEG-IFN-α) and orally administered nucleos(t)ide anologs (NUC), such as tenofovir, entecavir, adefovir, telbivudine, and lamivudine. Neither treatment achieves a high rate of functional cure (sustained loss of HBsAg with or without seroconversion). In addition, IFN-based therapies are associated with many side effects, whereas NUC frequently require prolonged or possibly life-long therapy, and some are associated with a high risk of viral resistance.

Recently, a CpAM (core protein allosteric modulator) was developed for the treatment of patients with HBV infection. Compound (I) is a CpAM for treatment and/or prophylaxis in a human by targeting on HBV capsid which was disclosed in WO2015132276, the structure is shown below:

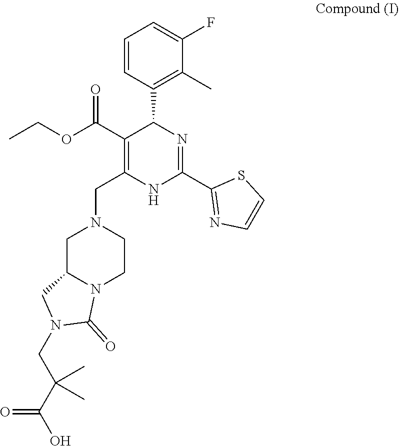 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]

Three clinical studies (Part 1 and 2 of YP39364 (NCT02952924), YP39406 (NCT03570658), and YP40218 (NCT03717064)) have been completed to characterize the safety, tolerability, pharmacokinetics (PK) and pharmacodynamics (PD) of Compound (I) in both healthy volunteers and CHB patients. Short-term PD markers (HBV DNA and HBV RNA) showed robust decline during four-week treatment with Compound (I), however significant decline of HBsAg has not been observed in such duration. Thus, there is an unmet medical need for a novel method of treatment with Compound (I) that could achieve a higher rate of functional cure with a finite treatment duration and excellent tolerability.

### SUMMARY OF THE INVENTION

In this invention, methods of Compound (I) monotherapy and Compound (I) in combination with other anti-HBV agents for 12-144 weeks were developed. Compound (I) is safe and well tolerated in chronic hepatitis B patients. Comparing with current NUC treatment, the current finite treatment methods of this invention minimize the risk of drug resistance and decrease adverse effects of NUC treatment (e.g. the risk of renal injury, bone density loss and creatine kinase elevation, etc).

In terms of efficacy, the treatment methods of this invention demonstrated a robust HBV DNA and HBV RNA decline in patients. Furthermore, treatment method of Compound (I) in combination with other anti-HBV agents for 12-144 weeks results in significant hepatitis B surface antigen (HBsAg) decline or loss. In addition, currently there is no effective antiviral treatment for immune tolerant patients (defined as patients with highly replicative (HBV DNA levels elevated) and low inflammatory (ALT levels are normal or without signs of significant inflammation or fibrosis by liver biopsy)). However, the method of treatment of this invention with Coumpound (I) can also induce robust HBV DNA and HBV RNA decline in such patients.

### DETAILED DESCRIPTION OF THE INVENTION

### Definitions

The term “NUC” denotes nucleos(t)ide analogs used as HBV therapy, including, but not limited to, lamivudine, adefovir dipivoxil, entecavir (ETV), telbivudine, tenofovir disoproxil fumarate (TDF) and tenofovir alafenamide (TAF).

The term “HBsAg loss” denotes quantitative HBsAg lower than limit of quantification (<0.05 IU/mL) measured by Elecsys® HBsAg II (Roche).

The term “fasted” denotes administration without meals for a period, which can be any time within 12 hours before next meal or after last meal.

The term “food” denotes any kind of food, including but not limited to, high fat food, light food or standard food.

The term “IFN-α” denotes conventional interferon-α or pegylated interferon-α (PEG-IFN-α). Examples of IFN-α include, but not limited to, Pegasys® (Roche) and PEG-Intron® (Schering Corporation).

The term “subcutaneous injection” or “SC” means that the drug is delivered via a short needle into patient's tissue layer between the skin and the muscle, subcutaneous doses will be administered at any appropriate site, for example, the abdomen or upper thigh. For multiple doses, the administration site should be rotated.

The term “intravenous injection” or “IV” means that the drug is delivered directly into patient's vein using a needle or tube.

The term “TLR agonist” means a class of toll-like receptor agonists which can boost antiviral cytokine production and activation of natural killer cells, B cells, and T cells, and these immune responses may play a role in the achievement of functional cure of CHB (i.e., HBsAg loss). Example of TLR agonist includes, but not limited to, TLR 7 agonist GS-9620 (Gilead Sciences Incs), TLR 8 agonist GS-9688 (Gilead Sciences Incs), TLR 7 agonist JNJ-64794964 (Janssen Research & Development LLC) and TLR9 modulator AIC649 (AiCuris Anti-infective Cures GmbH).

The term “retinoic acid-inducible Gene I” or “RIG-I” denotes a cytosolic pathogen recognition receptor that initiates the immune response against many RNA viruses. RIG-I modulator denotes a molecular which can induce type III and type I interferons through retinoic acid-inducible gene-I (RIG-I)-mediated sensing of the 5′-ε region of HBV pregenomic RNA. Example of RIG-I modulator includes, but not limited to, Inarigivir (SB9200) from Spring Bank Pharmaceuticals. (Sato S, Li K, Kameyama T, et al. The RNA sensor RIG-I dually functions as an innate sensor and direct antiviral factor for hepatitis B virus. Immunity 2015; 42: 123-32).

The term “small interfering RNA” or “siRNA” denotes small interfering ribonucleic acid, which is a class of double-stranded RNA molecules. siRNA targets any viral transcript and induce its degradation by the RISC/Ago2 complex, resulting in gene silencing. Examples of siRNA includes, but not limited to, ARB-1467 (Arbutus Biopharma), ARO-HBV (Arrowhead Pharma), AB-729 (Arbutus Biopharma), DCR-HBVS (Dicerna), Vir-2218 (Alnylam and Vir Biotech), BB-103 (Benitec) and Lunar-HBV (Arcturus, USA with Janssen).

The term “HBV LNA” denotes a molecule which is a N-Acetylgalactosamine (GalNAc)-targeted locked nucleic acid (LNA)-containing single stranded oligodeoxyribonucleotide, complementary to hepatitis B virus (HBV) genome-derived mRNA species, intended for the treatment of CHB infections.

The term “HBsAg inhibitor” denotes molecules which can interfere with the assembly/release, production or entry of HBsAg. Examples of HBsAg inhibitor include, but not limited to, REP 2139 (Replicor) and REP 2165 (Replicor), and Myrcludex B (MYR Pharma).

The term “HBV therapeutic vaccine” or “HBV vaccine” denotes a molecule which can stimulate or boost the host immune response to restore immune control, resulting in sustained suppression of HBV replication and ultimately HBsAg loss. Examples of HBV vaccine include, but not limited to, ABX203 (Center for Genetic Engineering and Biotechnology), INO-1800 (Inovio), HB-110 (Ichor Medical Systems with Janssen), TG1050 (Transgene) and HepTcell (Altimmune).

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### COMPOSITIONS OF TREATMENT

Another embodiment provides pharmaceutical compositions or medicaments containing the compounds of the invention and a therapeutically inert carrier, diluent or excipient, as well as methods of using the compounds of the invention to prepare such compositions and medicaments. In one example, Compound (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a Compound (I) is formulated in an acetate buffer, at pH 5. In another embodiment, the Compound (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to be potent as a CpAM, which can be used, but not limited, for the treatment or prevention of hepatitis B virus infected patients.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

### METHOD OF TREATMENT

This invention is related to (i) a method of treating HBV infection in a human patient, comprising administering to said patient a pharmaceutical composition containing an active ingredient of compound (I) in an amount from 200 mg QD or BID to 1000 mg QD or BID for 12-144 weeks with or without other anti-HBV agents; wherein compound (I) is 3[(8aS)-7-[[(4S)-5-ethoxycarbonyl-4-(3-flu oro-2-methyl-phenyl)-2-thiazol-2-yl-1,4-dihydropyrimidin-6-yl ]methyl]-3-oxo-5,6,8,8a-tetrahydro-1H-imidazo[1,5-a]pyra zin-2-yl]-15 2,2-dimethyl-propanoic acid.

A further embodiment of present invention is (ii) the method according to (i), wherein compound (I) is administered via oral, SC or IV.

A further embodiment of present invention is (iii) the method according to (i) or (ii), wherein compound (I) is administered fasted or with food.

A further embodiment of present invention is (iv) the method according to any one of (i) to (iii), wherein the amount and frequency of administration of compound (I) is 200 mg QD or BID, 400 mg QD or BID, 600 mg QD or BID, 800 mg QD or BID, or 1000 mg QD or BID.

A further embodiment of present invention is (v) the method according to any one of (i) to (iv), wherein compound (I) is administered for 12 weeks, 24 weeks, 36 weeks, 48 weeks, 52 weeks, 60 weeks, 72 weeks, 84 weeks, 96 weeks, 104 weeks, 108 weeks, 120 weeks, 132 weeks, or 144 weeks.

A further embodiment of present invention is (vi) the method according to any one of (i) to (v), wherein compound (I) is administered without other anti-HBV agents.

A further embodiment of present invention is (vii) the method according to any one of (i) to (vi), wherein compound (I) is administered with one or two other anti-HBV agents, wherein the other anti-HBV agent is independently selected from NUC, IFN-α, TLR agonist, RIG-I modulator, siRNA, HBVLNA oligonucleotide, HBsAg inhibitor and HBV vaccine. The treatment method of other anti-HBV agent can be applied according to existed standard of care.

A further embodiment of present invention is (viii) the method according to any one of (i) to (vii), wherein compound (I) is administered with NUC; wherein the NUC is selected from ETV, TAF and TDF.

A further embodiment of present invention is (ix) the method according to any one of (i) to (viii), wherein compound (I) is administered for 48-96 weeks, particularly 48 weeks, 52 weeks, 60 weeks, 72 weeks, 84 weeks or 96 weeks.

A further embodiment of present invention is (x) the method according to any one of (i) to (ix), wherein compound (I) is administered at 600 mg QD via oral and fasted with NUC for 48 weeks; wherein NUC is selected from ETV, TAF and TDF.

A further embodiment of present invention is (xi) the method according to any one of (i) to (x), wherein compound (I) is administered with NUC and IFN-α; wherein NUC is selected from ETV, TAF and TDF.

A further embodiment of present invention is (xii) the method according to any one of (i) to (xi), wherein compound (I) is administered for 48-96 weeks, particularly 48 weeks, 52weeks, 60 weeks, 72 weeks, 84 weeks or 96 weeks.

A further embodiment of present invention is (xiii) the method according to any one of (i) to (xii), wherein compound (I) is administered at 600 mg QD via oral and fasted with NUC and IFN-α for 48 weeks; wherein NUC is selected from ETV, TAF and TDF; wherein the IFN-α is selected from Pegasys® and PEG-Intron®.

A further embodiment of present invention is (xiv) the method according to any one of (i) to (xiii), wherein compound (I) is administered with NUC and TLR agonist; wherein the NUC is selected from ETV, TAF and TDF; wherein the TLR agonist is selected from GS-9620, GS-9688, JNJ-64794964 and AIC649.

A further embodiment of present invention is (xv) the method according to any one of (i) to (xiv), wherein compound (I) is administered with NUC and RIG-I modulator; wherein the NUC is selected from ETV, TAF and TDF.

A further embodiment of present invention is (xvi) the method according to any one of (i) to (xv), wherein the RIG-I modulator is Inarigivir.

A further embodiment of present invention is (xvii) the method according to any one of (i) to (xvi), wherein compound (I) is administered for 12-48 weeks, particularly 12 weeks, 24 weeks, 36 weeks or 48 weeks.

A further embodiment of present invention is (xviii) the method according to any one of (i) to (xvii), wherein compound (I) is administered with NUC and siRNA; wherein the NUC is selected from ETV, TAF and TDF.

A further embodiment of present invention is (xix) the method according to any one of (i) to (xviii), wherein the siRNA is selected from ARB-1467, ARO-HBV, AB-729, DCR-HBVS,

Vir-2218, BB-103 and Lunar-HBV.

A further embodiment of present invention is (xx) the method according to any one of (i) to (xix), wherein compound (I) is administered with NUC and HBV LNA oligonucleotide; wherein the NUC is selected from ETV, TAF and TDF; wherein the HBV LNA oligonucleotide is the compound (II), which is any one of the compounds disclosed in WO2015/173208 or WO2014/179627.

A further embodiment of present invention is (xxi) the method according to any one of (i) to (xx), wherein compound (I) is administered for 12-48 weeks, particularly 12 weeks, 24 weeks, 36 weeks or 48 weeks.

A further embodiment of present invention is (xxii) the method according to any one of (i) to (xxi), wherein compound (I) is administered with NUC and HBsAg inhibitor; wherein the NUC is selected from ETV, TAF and TDF; wherein the HBsAg inhibitor is selected from REP 2139, REP 2165 and Myrcludex B.

A further embodiment of present invention is (xxiii) the method according to any one of (i) to (xxii), wherein compound (I) is administered with another anti-HBV agent, wherein the anti-HBV agent is selected from Pegasys®, PEG-Intron®, Inarigivir, ARB-1467, ARO-HBV, AB-729, DCR-HBVS, Vir-2218, BB-103, Lunar-HBV, compound (II), REP 2139, REP 2165, Myrcludex B, GS-9620, GS-9688, JNJ-64794964, AIC649, ABX203, INO-1800, HB-110, TG1050 and HepTcell.

### EXAMPLES

The invention will be more fully understood by reference to the following examples. They should not, however, be construed as limiting the scope of the invention.

### ABBREVIATIONS

AASLD: American Association for the Study of the Liver

APASL: Asia-Pacific Association for the Study of the Liver

BID: twice a day

CHB: chronic hepatitis B

EASL: European Association for the Study of the Liver

ETV: Entecavir

NUC: nucleos(t)ide analog

HBeAg: hepatitis B e antigen

HBsAg: hepatitis B surface antigen

HBV: hepatitis B virus

HCC: hepatocellular carcinoma

PEG-IFN: pegylated interferon

QD: once a day

TAF: tenofovir alafenamide.

TDF: tenofovir disoproxil fumarate

WHO: World Health Organization

### GENERAL EXPERIMENTAL CONDITIONS

Study population: patients infected with HBV (Terrault NA, Lok ASF, McMahon BJ, et al. Update on prevention, diagnosis, and treatment of chronic hepatitis B: AASLD 2018 hepatitis B guidance. Hepatology. 2018; 67:1560-1599.European Association for the Study of the Liver. EASL 2017 Clinical Practice Guidelines on the management of hepatitis B virus infection. J Hepatol. 2017; 67:370-398.).

Compounds:

Compound (I): 3-[(8aS)-7-[[(4S)-5-ethoxycarbonyl-4-(3-fluoro-2-methyl-phenyl)-2-thiazol-2-yl-1,4-dihydropyrimidin-6-yl]me thyl]-3-oxo-5,6,8,8a-tetrahydro-1H-imidazo[1,5-alpyrazin-2-yl]-15 2,2-dimethyl-propanoic acid, which is an HBV CpAM with structure shown below:

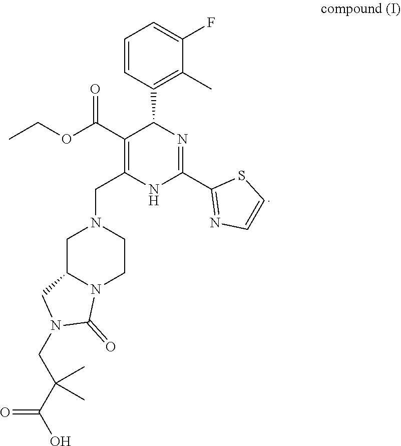 [SMILES: CCOC(=O)C1=C(CN2CCN3C(=O)N(CC(C)(C)C(=O)O)C[C@@H]3C2)NC(c2nccs2)=N[C@H]1c1cccc(F)c1C]

Compound (II): an HBV LNA oligonucleotide which is any one of the compounds disclosed in WO2015/173208 or WO2014/179627.

Inarigivir: a small molecule dinucleotide orally active HBV antiviral developed by Spring Bank Pharmaceuticals, which has both direct acting and immune modulatory activity, stimulating the host innate antiviral response. It binds to the cytosolic pattern recognition receptor retinoic-acid-inducible gene (RIG-I) to enhance RIG-I binding to the 5′-epsilon region of the HBV pregenomic RNA. (Walsh R., Hammond R., Jackson K., et al. Effects of SB9200 (Inarigivir) therapy on immune responses in patients with chronic hepatitis B. Journal of Hepatology 2018; 68 Supplement 1: S89-).

REP 2139 or REP 2165: a nucleic acid polymers developed by Replicor company. It can act via a post-entry mechanism to block the assembly/release of HBV subviral particles, allowing clearance of HBsAg from the circulation (Bazinet M., Pântea V., Placinta G., et al. Establishment of high rates of functional control and reversal of fibrosis following treatment of HBeAg negative chronic HBV infection with REP 2139-MG/REP 2165-MG, tenofovir disoproxil fumarate and pegylated interferon alpha-2a. Hepatology 2018; 68 Supplement 1:234A-235A).

GS-9620: a small molecule of TLR 7 agonist developed by Gilead Sciences Incs. (Agarwal K, Ahn S.H., Elkhashab M., Safety and efficacy of vesatolimod (GS-9620) in patients with chronic hepatitis B who are not currently on antiviral treatment. Journal of Viral Hepatitis 2018; 25:1331-1340).

GS-9688: a potent and selective small-molecule of TLR 8 agonist developed by Gilead Scientices Incs. (Khanam A., Yoon J.A., Poonia B.,et al. GS-9688, a toll-like receptor 8 agonist induces innate and adaptive antiviral immune response in chronic hepatitis B patients. Hepatology 2018;68 Supplement 1:329A-330A).

JNJ-64794964: an oral toll-like receptor-7 agonist aims to treat chronic HBV, which is currently developed by Janssen Research & Development LLC. (Ed Gane, Mina Pastagia, An De Creus, et al. A Phase 1, Double-Blind, Randomised, Placebo-Controlled, First-in-Human Study of the Safety, Tolerability, Pharmacokinetics and Pharmacodynamics of oral JNJ-64794964, a Toll-like Receptor-7 Agonist, in Healthy Adults. Journal of Hepatology 2019; 70 e141-e382: FRI-198).

AIC649: an inactivated parapoxvirus ovis particle preparation which has been shown to directly address the antigen presenting cell arm of the host immune defense leading to a regulated cytokine release and activation of T cell responses. AIC649 is developed by AiCuris Anti-infective Cures GmbH. (Ibironke Addy, Alen Jambrecina, Thomas Berg, et al. First in Human, single ascending dose clinical trial of AIC649 in patients with chronic hepatitis. Journal of Hepatology 2019; 70 e141-e382: FRI-199).

Myrcludex B: an anti-HBV molecule owned by MYR Pharma, which can bind and inactivate the hepatocyte surface protein sodium taurocholate co-transporting polypeptide, misdirect HBV to an unproductive pathway and thereby prevents an infection of the cell. (http://myr-pharma.com/science/about-myrcludex-b).

DCR-HBVS: an investigational drug in development in Dicerna Pharmaceuticals, Inc. This investigational drug is comprised of a single GalXC molecule that targets HBV messenger RNAs within the HBsAg gene sequence region.

HBV vaccine: ABX203 (Center for Genetic Engineering and Biotechnology), INO-1800 (Inovio), HB-110 (Ichor Medical Systems with Janssen), TG1050 (Transgene) or HepTcell (Altimmune).

General Methods: To explore the effects of Compound (I) on HBsAg loss or decline, Compound (I) is administered following the dose regimen listed in Table 1. The primary measure is the quantitative HBsAg decline or HBsAg loss (<0.05 IU/mL, measured by Elecsys® HBsAg II (Roche)®) at 24 weeks post treatment.

Study Design: The doses selected for Compound (I) and schedules utilized in this invention are summarized in Table 1 below:

TABLE 1
Dose regimen of Compound (I)

| Study | | Administration | Dietary | Combination | Treatment duration of |
|---|---|---|---|---|---|
| No. | Dose level | route/frequency | requirement | strategy | Compound (I) |
| 1 | 600 mg | Oral, QD | Fasted | Compound (I) in | 48 weeks |
| | | | | combination with | |
| | | | | NUC (ETV, TAF | |
| | | | | or TDF) | |
| 2 | 600 mg | Oral, QD | Fasted | Compound (I) in | 52 weeks, 60 weeks, 72 |
| | | | | combination with | weeks, 84 weeks or 96 |
| | | | | NUC (ETV, TAF | weeks |
| | | | | or TDF) | |
| 3 | 600 mg | Oral, QD | Fasted | Compound (I) in | 48 weeks |
| | | | | combination with | |
| | | | | NUC (ETV, TAF | |
| | | | | or TDF) and IFN-α | |
| | | | | (Pegasys ® or PEG- | |
| | | | | Intron ®) | |
| 4 | 600 mg | Oral, QD | Fasted | Compound (I) in | 52 weeks, 60 weeks, 72 |
| | | | | combination with | weeks, 84 weeks or 96 |
| | | | | NUC (ETV, TAF | weeks |
| | | | | or TDF) and IFN-α | |
| | | | | (Pegasys ® or PEG- | |
| | | | | Intron ®) | |
| 5 | 200 mg or | Oral, QD/BID; | Fasted or | Compound (I) | 12-144 weeks (e.g. 12 |
| | 400 mg or | SC, QD/BID; or | with food | monotherapy | weeks, 24 weeks, 36 |
| | 600 mg or | IV, QD/BID | | | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 6 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 7 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | IFN-α (Pegasys ® or | weeks, 48 weeks, 52 |
| | 800 mg or | | | PEG-Intron ®) | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 8 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and TLR | weeks, 60 weeks, 72 |
| | 1000 mg | | | agonist | weeks, 84 weeks, 96 |
| | | | | | weeks, 104 weeks, 108 |
| | | | | | weeks, 120 weeks, 132 |
| | | | | | weeks, or 144 weeks) |
| 9 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and GS- | weeks, 60 weeks, 72 |
| | 1000 mg | | | 9688 | weeks, 84 weeks, 96 |
| | | | | | weeks, 104 weeks, 108 |
| | | | | | weeks, 120 weeks, 132 |
| | | | | | weeks, or 144 weeks) |
| 10 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and GS- | weeks, 60 weeks, 72 |
| | 1000 mg | | | 9620 | weeks, 84 weeks, 96 |
| | | | | | weeks, 104 weeks, 108 |
| | | | | | weeks, 120 weeks, 132 |
| | | | | | weeks, or 144 weeks) |
| 11 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and JNJ- | weeks, 60 weeks, 72 |
| | 1000 mg | | | 64794964 | weeks, 84 weeks, 96 |
| | | | | | weeks, 104 weeks, 108 |
| | | | | | weeks, 120 weeks, 132 |
| | | | | | weeks, or 144 weeks) |
| 12 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and | weeks, 60 weeks, 72 |
| | 1000 mg | | | AIC649 | weeks, 84 weeks, 96 |
| | | | | | weeks, 104 weeks, 108 |
| | | | | | weeks, 120 weeks, 132 |
| | | | | | weeks, or 144 weeks) |
| 13 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and RIG-I | weeks, 60 weeks, 72 |
| | 1000 mg | | | modulator | weeks, 84 weeks, 96 |
| | | | | | weeks, 104 weeks, 108 |
| | | | | | weeks, 120 weeks, 132 |
| | | | | | weeks, or 144 weeks) |
| 14 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and | weeks, 60 weeks, 72 |
| | 1000 mg | | | Inarigivir | weeks, 84 weeks, 96 |
| | | | | | weeks, 104 weeks, 108 |
| | | | | | weeks, 120 weeks, 132 |
| | | | | | weeks, or 144 weeks) |
| 15 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and | weeks, 60 weeks, 72 |
| | 1000 mg | | | siRNA | weeks, 84 weeks, 96 |
| | | | | | weeks, 104 weeks, 108 |
| | | | | | weeks, 120 weeks, 132 |
| | | | | | weeks, or 144 weeks) |
| 16 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and | weeks, 60 weeks, 72 |
| | 1000 mg | | | Compound (II) | weeks, 84 weeks, 96 |
| | | | | | weeks, 104 weeks, 108 |
| | | | | | weeks, 120 weeks, 132 |
| | | | | | weeks, or 144 weeks) |
| 17 | 200 mg or | Oral, QD/BID | Fasted or | Compound (I) in | 12-48 weeks (e.g. 12 |
| | 400 mg or | | with food | combination with | weeks, 24 weeks, 36 |
| | 600 mg or | | | NUC (ETV, TAF | weeks or 48 weeks) |
| | 800 mg or | | | or TDF) and | |
| | 1000 mg | | | Compound (II) | |
| 18 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and DCR- | weeks, 60 weeks, 72 |
| | 1000 mg | | | HBVS | weeks, 84 weeks, 96 |
| | | | | | weeks, 104 weeks, 108 |
| | | | | | weeks, 120 weeks, 132 |
| | | | | | weeks, or 144 weeks) |
| 19 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and | weeks, 60 weeks, 72 |
| | 1000 mg | | | HBsAg inhibitor | weeks, 84 weeks, 96 |
| | | | | (REP 2139 or REP | weeks, 104 weeks, 108 |
| | | | | 2165) | weeks, 120 weeks, 132 |
| | | | | | weeks, or 144 weeks) |
| 20 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | NUC (ETV, TAF | weeks, 48 weeks, 52 |
| | 800 mg or | | | or TDF) and | weeks, 60 weeks, 72 |
| | 1000 mg | | | Myrcludex B | weeks, 84 weeks, 96 |
| | | | | | weeks, 104 weeks, 108 |
| | | | | | weeks, 120 weeks, 132 |
| | | | | | weeks, or 144 weeks) |
| 21 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | RIG-I modulator | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 22 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | Inarigivir | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 23 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | siRNA | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 24 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | Compound (II) | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 25 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | DCR-HBVS | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 26 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | REP 2139 or REP | weeks, 48 weeks, 52 |
| | 800 mg or | | | 2165 | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 27 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | Myrcludex B | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 28 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | TLR agonist | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 29 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | GS-9688 | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 30 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | GS-9620 | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 31 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | JNJ-64794964 | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 32 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | AIC649 | weeks, 48 weeks, 52 |
| | 800 mg or | | | | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |
| 33 | 200 mg or | Oral, QD/BID; | Fasted or | Compound (I) in | 12-144 weeks (e.g. 12 |
| | 400 mg or | SC, QD/BID; or | with food | combination with | weeks, 24 weeks, 36 |
| | 600 mg or | IV, QD/BID | | therapeutic HBV | weeks, 48 weeks, 52 |
| | 800 mg or | | | vaccine | weeks, 60 weeks, 72 |
| | 1000 mg | | | | weeks, 84 weeks, 96 |
| | | | | | weeks, 104 weeks, 108 |
| | | | | | weeks, 120 weeks, 132 |
| | | | | | weeks, or 144 weeks) |

1. A method of treating HBV infection in a human patient, comprising administering to said patient a pharmaceutical composition containing an active ingredient of compound (I) in an amount from 200 mg QD or BID to 1000 mg QD or BID for 12-144 weeks with or without other anti-HBV agent; wherein compound (I) is 3-[(8aS)-7-[[(4S)-5-ethoxycarbonyl-4-(3-fluoro-2-methyl-phenyl)-2-thiazol-2-yl-1,4-dihydropyrimidin-6-yl]methyl]-3-oxo-5,6,8,8a- tetrahydro-1H-imidaz[1,5-a]pyrazin-2-yl]-15 2,2-dimethyl-propanoic acid.

2. The method according to claim 1, wherein compound (I) is administered via oral, SC or IV.

3. The method according to claim 2, wherein compound (I) is administered fasted or with food.

4. The method according to any one of claims 1 to 3, wherein the amount and frequency of administration of compound (I) is 200 mg QD or BID, 400 mg QD or BID, 600 mg QD or BID, 800 mg QD or BID, or 1000 mg QD or BID.

5. The method according to any one of claims 1 to 4, wherein compound (I) is administered for 12 weeks, 24 weeks, 36 weeks, 48 weeks, 52 weeks, 60 weeks, 72 weeks, 84 weeks, 96 weeks, 104 weeks, 108 weeks, 120 weeks, 132 weeks, or 144 weeks.

6. The method according to claim 5, wherein compound (I) is administered without other anti-HBV agent.

7. The method according to claim 5, wherein compound (I) is administered with one or two other anti-HBV agents, wherein the other anti-HBV agent is independently selected from NUC, IFN-α, TLR agonist, RIG-I modulator, siRNA, HBV LNA oligonucleotide, HBsAg inhibitor and HBV vaccine.

8. The method according to claim 7, wherein compound (I) is administered with NUC; wherein the NUC is selected from ETV, TAF and TDF.

9. The method according to claim 8, wherein compound (I) is administered for 48-96 weeks, particularly 48 weeks, 52 weeks, 60 weeks, 72 weeks, 84 weeks or 96 weeks.

10. The method according to claim 8 or 9, wherein compound (I) is administered at 600 mg QD via oral and fasted with NUC for 48 weeks; wherein NUC is selected from ETV, TAF and TDF.

11. The method according to claim 7, wherein compound (I) is administered with NUC and IFN-α; wherein NUC is selected from ETV, TAF and TDF.

12. The method according to claim 12, wherein compound (I) is administered for 48-96 weeks, particularly 48 weeks, 52weeks, 60 weeks, 72 weeks, 84 weeks or 96 weeks.

13. The method according to claim 11 or 12, wherein compound (I) is administered at 600 mg QD via oral and fasted with NUC and IFN-α for 48 weeks; wherein NUC is selected from ETV, TAF and TDF; wherein the IFN-α is selected from Pegasys® and PEG-Intron®.

14. The method according to claim 7, wherein compound (I) is administered with NUC and TLR agonist; wherein the NUC is selected from ETV, TAF and TDF; wherein the TLR agonist is selected from GS-9620, GS-9688, JNJ-64794964 and AIC649.

15. The method according to claim 7, wherein compound (I) is administered with NUC and RIG-I modulator; wherein the NUC is selected from ETV, TAF and TDF.

16. The method according to claim 15, wherein the RIG-I modulator is Inarigivir.

17. The method according to claim 16, wherein compound (I) is administered for 12-48 weeks, particularly 12 weeks, 24 weeks, 36 weeks or 48 weeks.

18. The method according to claim 7, wherein compound (I) is administered with NUC and siRNA; wherein the NUC is selected from ETV, TAF and TDF.

19. The method according to claim 18, wherein the siRNA is selected from ARB-1467, ARO-HBV, AB-729, DCR-HBVS, Vir-2218, BB-103 and Lunar-HBV.

20. The method according to claim 7, wherein compound (I) is administered with NUC and HBV LNA oligonucleotide; wherein the NUC is selected from ETV, TAF and TDF; wherein the HBV LNA oligonucleotide is the compound (II).

21. The method according to claim 20, wherein compound (I) is administered for 12-48 weeks, particularly 12 weeks, 24 weeks, 36 weeks or 48 weeks.

22. The method according to claim 7, wherein compound (I) is administered with NUC and HBsAg inhibitor; wherein the NUC is selected from ETV, TAF and TDF; wherein the HBsAg inhibitor is selected from REP 2139, REP 2165 and Myrcludex B.

23. The method according to claim 7, wherein compound (I) is administered with another anti-HBV agent, wherein the anti-HBV agent is selected from Pegasys®, PEG-Intron®, Inarigivir, ARB-1467, ARO-HBV, AB-729, DCR-HBVS, Vir-2218, BB-103, Lunar-HBV, compound (II), REP 2139, REP 2165, Myrcludex B, GS-9620, GS-9688, JNJ-64794964, AIC649, ABX203, INO-1800, HB-110, TG1050 and HepTcell.
